(19) United States
(12) Patent Application Publication
(10) Pub. No.: US 2004/0015117 A1
(43) Pub. Date: Jan. 22, 2004

(54) Reflecting lower leg protector

(76) Inventor: Rejean Gauthier, St-Lin (CA)

Correspondence Address: INVENTARIUM, 4050 ROSEMONT BLVD, #1607, Montreal, QC, H1X 1M4, CA

(21) Appl. No.: 10/617,301

(22) Filed: Jul. 11, 2003

Related U.S. Application Data
(60) Provisional application No. 60/395,993, filed on Jul. 16, 2002.

(51) Int. Cl. A61F 13/00

(52) U.S. Cl. 602/62

(57) ABSTRACT

A reflecting lower leg protector is designed to be releasably attachable to the lower leg of a user with ease. Also, the proposed reflecting lower leg protector is provided with highly reflective material to enhance the visibility of the wearer and provide additional protection in situations where many users are working closely together in order to avoid any injury or to simply enhance visibility under varying degrees of illumination for any reason.

This application claims priority based on provisional aplication No. 60/395,993 filed Jul. 16, 2002

### FIELD OF THE INVENTION

The present invention relates to the general field of safety equipment but more particularly to leg protecting equipment.

### BACKGROUND OF THE INVENTION

Some activities involve a higher level of risks in the course of their execution, it is therefore advisable for anyone engaged in such activities to use some sort of protective equipment. Of particular concern here is the protection of lower legs when using weed cutting equipments.

Indeed, lawn mowing or more generally mowing of grasses and weeds can be hazardous for some type of equipment can throw small debris such as pebbles and twigs and other light objects at great speed over short distances.

Those familiar with weed wackers are familiar with the associated hazards, discomfort and inconvenience as they find their lower trousers covered with grass and sometimes feel the stinging of pebbles and other small projectiles. Besides the risks of injury and the stinging, the mere need of preventing the users pants from being soiled by wet grass is reason enough to wear some form of protective equipment.

Over the years, many leg protecting equipments have been proposed, in some instances leg protecors were developped for the needs of cyclists or for rain and snow, when made out of plastic materials; other leg protectors also cover the top of the foot all the way up to a point below the knee. Sponge-like rubber and leather constitute other materials from which leg protectors are made of and most of the prior art leg protectors make use of velcro to attach the leg protector to the lower leg of the user.

### SUMMARY OF THE INVENTION

The herein proposed reflecting lower leg protector is designed to be releasably attachable to the lower leg of a user with ease. Also, the proposed reflecting lower leg protector is provided with highly reflective material to enhance the visibility of the wearer and provide additional protection in situations where many users are working closely together in order to avoid any injury or to simply enhance visibility under varying degrees of illumination for any reason.

It is therefore an object of this invention to provide for an easily attachable and removable pair of leg protectors.

It is another object of this invention to provide for a leg protector which makes the pants of the user less prone to soilage.

It is yet another object of this invention to provide for a leg protector which protects the lower legs of the user to make it less prone to stings and injuries.

It is a final object of this invention to provide for a leg protector which makes the legs of the user more visible under any light conditions.

The foregoing and other objects, features, and advantages of this invention will become more readily apparent from the following detailed description of a preferred embodiment with reference to the accompanying drawings, wherein the preferred embodiment of the invention is shown and described, by way of examples. As will be realized, the invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF THE DRAWINGS

FIG. 1 In a front elevational view, illustrates a reflecting lower leg protector in accordance with an embodiment of the present invention in a flat configuration prior to use.

FIG. 2 In a perspective elevational view, illustrates the reflecting lower leg protector in use.

### DETAILED DESCRIPTION

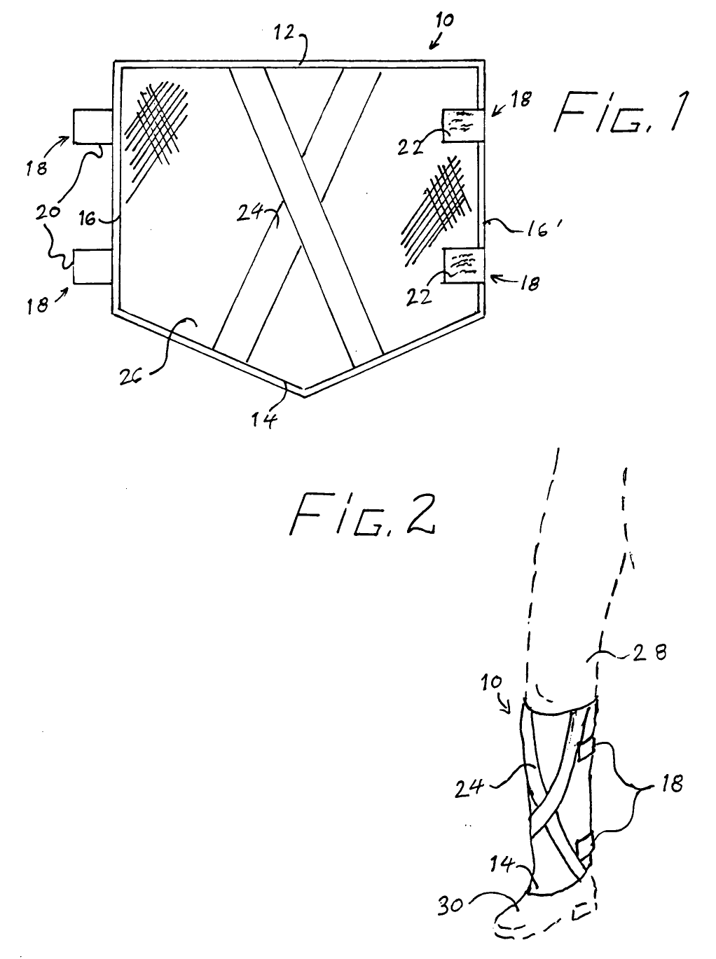

Referring to FIG. 1, there is shown a reflecting lower leg protector (10) in accordance with an embodiment of the present invention. The reflecting lower leg protector (10) is shown laying flat to better describe its components which include: a top part (12) and a bottom part (14); two lateral sides (16,16′) that are generally but not necessarily parallel to each other and the bottom part (14) which is in a generally pointed triangular configuration and is meant to cover the top of a user's foot. A set of hook and pile straps (18) subdivided into hooked ends (20) (on reverse side in this figure) and piled ends (22) are mounted on the lateral sides (16, 16′).

At the center of the reflecting lower leg protector (10) is a visible marker (24) which can be made in the shape of an “X” or any other type of strong visual indicia and made of a highly reflective fluorescent type material which is highly visible even under less than ideal conditions such as dark overcast day, rainy days and even at night by reflecting light from artificial sources. The marker (24) can be incorporated into the reflecting lower leg protector (10) such as by being dyed in, painted on or affixed using any suitable means such as sewing, glueing, melting or any combination thereof. The surface (26) of the reflecting lower leg protector (10) is made of a strong and resillient material suitable for protecting against projectiles such as flung debris, twigs and pebbles and is also preferably of a highly visible and reflective color to be visible under any light condition.

Referring to FIG. 2, the reflecting lower leg protector (10) in use on the lower part of a leg (28) (shown in ghost lines) by fastening the hook and pile straps (18) around the lower leg and placing the bottom part (14) so as to cover the top of the user's foot (30).

1. A reflecting lower leg protector to protect the lower leg and top of foot comprising:
a top part, a bottom part and two lateral sides and the bottom part being in a generally pointed triangular configuration so as to cover the top of a user's foot; a set of hook and pile straps subdivided into hooked ends and piled ends mounted on the lateral sides;
a visible marker with a strong visual indicia made of a highly reflective fluorescent type material highly visible under any light condition.

2. A reflecting lower leg protector as in claim 1 wherein:
said visible marker is in the shape of an “X”.

3. A reflecting lower leg protector as in claim 1 wherein:
said reflecting lower leg protector using strong and resillient material suitable for protecting against projectiles.

4. A reflecting lower leg protector as in claim 1 having the following method of use:
a user fastens the hook and pile straps around the lower leg and places the bottom part the top of his foot.
